(19) United States
(12) Patent Application Publication
(10) Pub. No.: US 2013/0085121 A1
(43) Pub. Date: Apr. 4, 2013

(54) PHARMACEUTICAL COMPOSITIONS COMPRISING PHOSPHATE BINDER, CALCIUM RECEPTOR-ACTIVE COMPOUND AND/OR ACTIVE VITAMIN D

(71) Applicants: Jianguo Wang, Brampton (CA); Yuehua Sun, Brampton (CA)

(72) Inventors: Jianguo Wang, Brampton (CA); Yuehua Sun, Brampton (CA)

(21) Appl. No.: 13/614,485

(22) Filed: Sep. 13, 2012

Related U.S. Application Data
(60) Provisional application No. 61/541,288, filed on Sep. 30, 2011.

(51) Int. Cl. A61K 31/136; A61J 3/10; A61K 31/592; A61K 31/59; A61K 31/593

(52) U.S. Cl. 514/167; 514/654; 264/122

(57) ABSTRACT

The present invention is an oral solid pharmaceutical compositions for the treatment of kidney diseases and mineral bone disorder including a phosphate binder, a calcium receptor-active compound and at least one pharmaceutically acceptable excipient, the invention further including a method for preparing the pharmaceutical compositions including the steps of granulating cinacalcet and/or sevelamer and/or vitamin D by one of a wet and a dry granulation process, each with at least one pharmaceutically acceptable excipient to form cinacalcet granules and/or sevelamer granules and/or vitamin D granules, mixing at least two of the cinacalcet granules, sevelamer granules and vitamin D granules to form a granules mixture and compressing the granules mixture to tablets or encapsulating the granules mixture into capsules or pulverizing the granules mixture into a dispersion powder.

### BACKGROUND OF THE INVENTION AND DESCRIPTION OF RELATED ART

The present invention is directed towards new pharmaceutical compositions and methods of making the compositions comprising combinations of vitamin D (including vitamin D analogs and active vitamin D) and/or sevelamer and/or cinacalcet in oral solid forms having entirely unexpected excellent stability, content uniformity, dissolution and bioavailability. The compositions of the invention provide pharmaceutically effective doses of the active constituent compounds, for example, to effectively treat secondary hyperparathyroidism, treat elevated phosphate and PTH levels and treat chronic kidney disease. Patients suffering chronic kidney disease—mineral and bone disorder have had to typically take multiple medications per day, as well as several times per day. The pharmaceutical compositions of the present invention provide the advantages of optimal treatment and management of chronic kidney disease patients by reducing pill burden and improving patient compliance.

Patients with chronic kidney disease (CKD) develop changes in circulating blood levels of calcium and phosphorus. The kidney gradually loses the ability to remove phosphorus from the blood and cannot activate adequate amounts of vitamin D, to maintain normal levels of calcium. The parathyroid gland senses these changes and compensates to increase calcium by elevating production and release of parathyroid hormone (PTH). These metabolic changes alter bone metabolism to release calcium and accordingly lead to bone abnormalities including altered bone production. Consequently bone deformation, bone pain, and altered risks of fracture may occur. Those various bone mineral disorders, have recently been named CKD mineral and bone disorder (CKD-MBD). FIG. 1 illustrates the typical consequences and clinical syndrome of CKD-MBD that lead to morbidity, mortality, and decreased quality of life in the patients (Komaba et al., Inter Med, 47 (2008), p 989-994 and Ogata et al., Clin Exp Nephrol, 11 (2007) 11:261-268).

Three kinds of medicines, calcium-sensing receptors, phosphate binders, and vitamin D analogs, have been widely used to control PTH, calcium and phosphate levels in chronic kidney disease patients.

Calcium receptor-active compounds, such as cinacalcet hydrochloride, have been developed for treatment of secondary hyperparathyroidism (sHPT) that is critical in the management of CKD-MBD. These agents enhance the sensitivity of the parathyroid calcium-sensing receptors, thereby reducing levels of PTH, serum calcium and phosphorus, and calcium-phosphorus product (Block et al., N Engl J Med, 350 (2004), p 1516-1525). Cinacalcet is a drug that acts as a calcimimetic. The registered trademark is Sensipar® in the United States and Mimpara® in Europe by Amgen. It is a small molecule medicine used in treating CKD patients on dialysis who produce too much parathyroid hormone, a condition known as secondary hyperparathyroidism. In 2004, Sensipar® was approved in the United States and Europe for the treatment of secondary hyperparathyroidism in CKD patients on dialysis and for the treatment of hypercalcemia in patients with parathyroid carcinoma. In 2008, Mimpara® was approved in Europe for the reduction of hypercalcemia in patients with primary hyperparathyroidism where a parathyroidectomy is not clinically appropriate or is contraindicated. U.S. Pat. No. 6,011,068 and U.S. Pat. No. 6,031,003 disclose calcium receptor-active molecules, such as those having the general structure of cinacalcet. U.S. Pat. No. 6,211,244 and U.S. Pat. No. 6,001,884 disclose calcium receptor-active compounds related to cinacalcet and methods of preparing such compounds. U.S. Pat. No. 6,313,146 discloses agents that relate to the different roles of inorganic ion receptors in cellular and body processes. U.S. Pat. No. 7,829,595 discloses a pharmaceutical composition comprising a therapeutically effective amount of a calcium receptor-active compound and at least one pharmaceutically acceptable excipient, wherein the composition has a controlled dissolution profile. The patent further relates to a method of manufacturing the pharmaceutical composition, as well as a method of treating a disease using the pharmaceutical composition.

Phosphate binders, especially organic polymers such as sevelamer hydrochloride, have been demonstrated to decrease both phosphate and PTH levels without inducing hypercalcemia in dialysis patients (Am J Kidney Dis 42:S1-S202, 2003 (suppl 3)). Management of the phosphorus concentration is one of the primary treatments for CKD-MBD using phosphate binders to reduce serum phosphate concentration. Sevelamer is marketed under the brand name Renagel® (hydrochloric acid formulation) and Renvela® (Carbonate formulation) by Genzyme. The polymers prepared using the methods disclosed in U.S. Pat. Nos. 5,496,545, 5,667,775, 6,083,495, 6,509,013 and 6,858,203. U.S. Pat. No. 6,132,706 discloses methods of medical treatment for excess phosphate using guanidine-containing polymers. U.S. Pat. Nos. 6,383,518 and 6,697,087, disclose phosphate-binding polymer preparations. U.S. Pat. No. 7,014,846 discloses phosphate-binding polymers for oral administration.

Vitamin D analogs have been used to suppress PTH synthesis and secretion in patients with sHPT (Ogata et al., Ther Apher Dial, 10 (2006), p 355-64). Recent advances in vitamin D research have increased the understanding that in addition to the traditional role vitamin D has on calcium/phosphorus homeostasis and bone health, it also has a much broader role on many human physiological processes. There is a high prevalence of vitamin D deficiency in the chronic kidney disease population. Vitamin D is often administered to patients to mitigate detrimental effects on bone health and mineral metabolism. Despite potential increases in serum calcium and phosphorus due to activation of vitamin D receptors in the gut, vitamin D administration has been associated with a survival benefit to CKD patients.

The benefits of concomitant administration of cinacalcet, sevelamer, and vitamin D analogs have been demonstrated. Recently in Japan, a prospective study (Patient Survival and the Combination of Vitamin D and Phosphorus Binder in Dialysis, Dialysis & Transplantation, October 2010, p 427-431) showed for the first time that a combination of vitamin D and a phosphorus binder was significantly associated with prognosis in Japanese dialysis patients. It is concluded that the combination of vitamin D and phosphate binder was significantly related to survival in dialysis patients.

However it has been a challenge to adequately control Kidney Disease Outcomes Quality Initiative (KDOQI™) biochemical targets for chronic kidney disease, bone and mineral disorder (CKD-MBD), which include parathyroid hormone (PTH), calcium, and phosphate. Table 1 and Table 2 list the frequency of measurements and ranges for PTH, phosphorus, and calcium recommended by KDOQI clinical guidelines for bone metabolism and disease in CKD (Am J Kidney Dis 42:S1-S202, 2003 (suppl 3)).

TABLE 1
Frequency of Measurement of PTH and Calcium/Phosphorus by Stage of CKD

| | | Measurement of |
|---|---|---|
| CKD Stage | Measurement of PTH | Calcium/Phosphous |
| 3 | Every 12 months | Every 12 months |
| 4 | Every 3 months | Every 3 months |
| 5 | Every 3 months | Every month |

TABLE 2
Range of PTH, Phosphorus, and Calcium Levels by Stage of CKD

| | Target “intact” PTH | Phosphous (mg/dL. | Corrected |
|---|---|---|---|
| CKD Stage | (pg/mL; pmol/L) | mmol/L) | total calcium |
| 3 | 35-70; 3.85-7.7 | 2.7-4.6; 0.87-1.49 | Normal* |
| 4 | 70-110; 7.7-12.1 | 2.7-4.6; 0.87-1.49 | Normal* |
| 5 | 150-300; 16.5-33.0 | 3.5-5.5; 1.13-1.78 | Normal* |

*Normal values for serum total calcium concentration vary among clinical laboratories, depending on the methods of measurement, with a normal range being 8.6 to 10.3 mg/dL (2.15 to 2.57 mmol/L) for adults.

In line with the KDOQI guidelines, the Japanese Society for Dialysis Therapy (JSDT) published original guidelines entitled: Guidelines for the management of secondary hyperparathyroidism in chronic dialysis patients (J Jpn Soc Dial Ther 2006; 39:1435-55). These guidelines set stringent targets for the concentrations of adjusted calcium at 8.4 to 10.0 mg/dL, phosphorus at 3.5 to 6.0 mg/dL, and intact PTH 60 to 180 μg/m L.

It was reported that among subjects with moderate to severe sHPT undergoing haemodialysis, concomitant administration with cinacalcet and low doses of vitamin D sterols improved achievement of the biochemical targets for CKD-MBD recommended by the KDOQI™ guidelines (Block et al., Nephrol Dial Transplant, 23 (2008), p 2311-2318). It was also reported that combined therapy with cinacalcet and low-dose active vitamin D derivatives improves control of PTH, calcium and phosphate in hemodialysis patients with sHPT and increases the likelihood of achieving KDOQI™ targets (Chertow et al., Clin J Am Soc Nephrol, 1 (2006), p 305-312).

The CKD-MBD patients typically take not only multiple medications a day but also several times a day. This dose complexity significantly reduces patient compliance in taking the medication for the treatment. Since the three kinds of medicines, calcium receptor-active compounds, phosphate binders and vitamin D analogs can be administrated concomitantly, the formulations comprised of different active agents as solid doses has the advantages for optimal treatment and management of CKD patients by reducing pill burden and improving patient compliance.

None of the foregoing patents and publications describe an oral solid composition comprising combined vitamin D and sevelamer, or vitamin D and cinacalcet, or sevelamer and cinacalcet, or combined three active compounds of vitamin D, sevelamer and cinacalcet, or their pharmaceutically acceptable salts thereof.

### SUMMARY OF THE INVENTION

The present invention includes composition embodiments that contain constituents of vitamin D, vitamin D analogs or active vitamin D analogs (such as 1,25-dihydroxy-vitamin D), sevelamer and cinacalcet or their pharmaceutically acceptable salts, in suitable combinations. Reference to vitamin D includes vitamin D analogs and active vitamin D.

For example, the compositions that include vitamin D and sevelamer combinations, vitamin D and cinacalcet combinations, sevelamer and cinacalcet combinations and combinations with all three active compounds (vitamin D, sevelamer and cinacalcet), are made into oral solid formulations that have particularly advantageous properties of excellent stability, content uniformity, dissolution and bioavailability.

Cinacalcet is a calcium receptor-active compound that is used to treat secondary hyperparathyroidism, which is critical to managing chronic kidney disease and mineral and bone disorder patients. Further, cinacalcet acts as a calcimimetic and in addition to being used to treat secondary hyperparathyroidism, especially in CKD patients on dialysis, it is used to treat hypercalcemia in patients with parathyroid carcinoma.

Sevelamer is a phosphate binder that decreases both phosphate and parathyroid hormone (PTH) levels without inducing hypercalcemia in dialysis patients. Management of the phosphorus concentration is one of the primary treatments for chronic kidney disease—mineral bone disorder (CKD-MBD) using phosphate binders to reduce serum phosphate concentration. Sevelamer is thus used to treat chronic kidney disease and particularly chronic kidney disease—mineral bone disorder.

Vitamin D analogs suppress PTH synthesis and secretion in patients with secondary hyperparathyroidism (sHPT). Vitamin D treats and maintains calcium/phosphorus homeostasis and bone health and further helps treat CKD patients to mitigate detrimental effects on bone health and mineral metabolism. Despite potential increases in serum calcium and phosphorus due to activation of vitamin D receptors in the gut, vitamin D administration provides a survival benefit to CKD patients.

CKD-MBD patients typically take not only multiple medications per day but also several times per day. This dose complexity significantly reduces patient compliance in taking the medication for the treatment. The present composition embodiments provide the three kinds of medicines, 1) calcium receptor-active compounds (Cinacalcet), 2) phosphate binders (Sevelamer) and 3) vitamin D, that are administrated concomitantly as a solid, oral composition. The compositions comprised of any combination the three different active agents as solid doses advantageously provide optimal treatment and management of CKD patients and CKD-MBD patients by reducing pill burden and improving patient compliance as well as by providing oral, solid dose compositions having excellent stability, content uniformity, dissolution, bioavailability and being easy to swallow.

The invention thus provides several embodiments including oral, solid dose compositions comprising pharmacologically effective amounts of vitamin D and/or sevelamer and/or cinacalcet, or pharmaceutically acceptable salts thereof, and comprising at least one pharmaceutically acceptable excipient. The composition has excellent stability, content uniformity, dissolution, bioavailability and is easy to swallow.

One embodiment of the present invention is an oral solid composition comprising pharmacologically effective amounts of vitamin D and sevelamer, or pharmaceutically acceptable salts thereof, and comprising at least one pharmaceutically acceptable excipient.

One embodiment of the present invention is an oral solid composition comprising pharmacologically effective amounts of vitamin D and cinacalcet, or pharmaceutically acceptable salts thereof, and comprising at least one pharmaceutically acceptable excipient.

One embodiment of the present invention is an oral solid composition comprising pharmacologically effective amounts of cinacalcet and sevelamer, or pharmaceutically acceptable salts thereof, and comprising at least one pharmaceutically acceptable excipient.

One embodiment of the present invention is an oral solid composition comprising pharmacologically effective amounts of vitamin D, sevelamer and cinacalcet, or pharmaceutically acceptable salts thereof, and comprising at least one pharmaceutically acceptable excipient.

In the inventive compositions comprising pharmacologically effective amounts of vitamin D and the active compounds sevelamer and/or cinacalcet, the content of vitamin D (as an active compound) is much less than other active compounds. The compositions of the present invention are constituted to advantageously provide excellent content uniformity and dissolution.

Accordingly, embodiments of the invention include methods and techniques that solve problems of content uniformity and dissolution by including, for example, the step of mixing separately granulated vitamin D, with the other active compounds. Cinacalcet and/or sevelamer can be added directly or as granules in the mixing step.

The oral, solid composition according to the present invention includes any solid pharmaceutically acceptable form such as a tablet, a capsule and the like. More preferably, the oral solid composition according to the invention is in the form of a tablet.

Additionally, the present invention provides oral solid compositions for treating chronic kidney disease, by administering the composition to a patient, up to 3-5 times a day.

The invention also provides the dose combinations of vitamin D, sevelamer and cinacalcet in the manufacture of oral solid compositions for the treatment of chronic kidney disease in different stages of the disease.

Additional aspects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and do not restrict the invention, as claimed.

### BRIEF DESCRIPTION OF THE DRAWINGS

FIG. 1 shows a schematic representation of the concept of CKD-MBD.

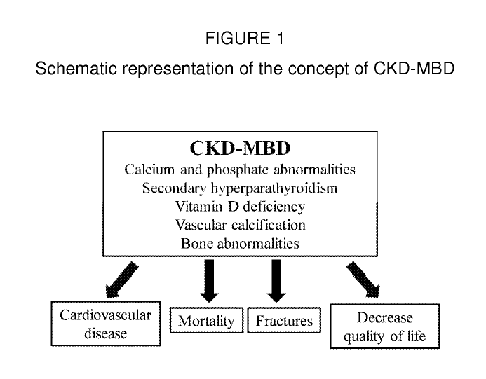

### DETAILED DESCRIPTION OF THE INVENTION

Embodiments of the present invention include the following:

1) An oral solid pharmaceutical composition for the treatment of kidney diseases and mineral bone disorder comprising:

a phosphate binder;

a calcium receptor-active compound; and

at least one pharmaceutically acceptable excipient.

2) The oral solid pharmaceutical composition of 1) wherein,

the phosphate binder is sevelamer or its pharmaceutically acceptable salt; and

the calcium receptor-active compound is cinacalcet or its pharmaceutically acceptable salt.

3) The oral solid pharmaceutical composition of 2) wherein,

the sevelamer is sevelamer hydrochloride or sevelamer carbonate in a dose of 400 mg to 2000 mg; and

the cinacalcet is cinacalcet hydrochloride in a dose of 5 mg to 90 mg.

4) The oral solid pharmaceutical composition of 2) wherein,

the sevelamer is sevelamer hydrochloride or sevelamer carbonate in a dose of 400 mg or 800 mg; and

the cinacalcet is cinacalcet hydrochloride in a dose selected from the group consisting of 5 mg, 10 mg, 15 mg, 30 mg, 60 mg, and 90 mg.

5) The oral solid pharmaceutical composition of 1) wherein,

the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

6) An oral solid pharmaceutical composition for the treatment of kidney diseases and mineral bone disorder comprising:

vitamin D;

a calcium receptor-active compound; and

at least one pharmaceutically acceptable excipient.

7) The oral solid pharmaceutical composition of 6) wherein,

the calcium receptor-active compound is cinacalcet or its pharmaceutically acceptable salt; and

the vitamin D is vitamin D or vitamin D₃, D₂ or active vitamin D selected from the group consisting of alfacalcidol, calcitriol, falecalcitol, maxacalcitol, and paricalcitol.

8) The oral solid pharmaceutical composition of 7) wherein,

the cinacalcet is cinacalcet hydrochloride in a dose of 5 mg to 180 mg; and

the vitamin D is in a dose of 0.1 μg to 100 μg.

9) The oral solid pharmaceutical composition of 7) wherein,

the cinacalcet is cinacalcet hydrochloride in a dose selected from the group consisting of 30 mg, 60 mg and 90 mg; and

the vitamin D is active vitamin D in a dose selected from the group consisting of 0.125 μg, 0.25 μg, 0.5 μg, 1 μg and 2 μg, or is vitamin D in a dose of 1-100 μg.

10) The oral solid pharmaceutical composition of 6) wherein,

the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

11) An oral solid pharmaceutical composition for the treatment of kidney diseases and mineral bone disorder comprising:

vitamin D;

a phosphate binder; and

at least one pharmaceutically acceptable excipient.

12) The oral solid pharmaceutical composition of 11) wherein,

the phosphate binder is sevelamer or its pharmaceutically acceptable salt; and

the vitamin D is vitamin D or vitamin D₃, D₂ or active vitamin D selected from the group consisting of alfacalcidol, calcitriol, falecalcitol, maxacalcitol, and paricalcitol.

13) The oral solid pharmaceutical composition of 12) wherein,

the sevelamer is sevelamer hydrochloride or sevelamer carbonate in a dose of 400 mg to 2000 mg; and

the vitamin D is in a dose of 0.1 μg to 100 μg.

14) The oral solid pharmaceutical composition of 12) wherein,

the sevelamer is sevelamer hydrochloride or sevelamer carbonate in a dose of 400 mg to 800 mg; and

the vitamin D is active vitamin D in a dose selected from the group consisting of 0.125 μg, 0.25 μg, 0.5 μg, 1 μg and 2 μg, or is vitamin D in a dose of 1-100 μg.

15) The oral solid pharmaceutical composition of 11) wherein,

the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

16) An oral solid pharmaceutical composition for the treatment of kidney diseases and mineral bone disorder comprising:

vitamin D;

a phosphate binder;

a calcium receptor-active compound; and

at least one pharmaceutically acceptable excipient.

17) The oral solid pharmaceutical composition of 16) wherein,

the vitamin D is vitamin D, vitamin D₃, D₂ or active vitamin D selected from the group consisting of alfacalcidol, calcitriol, falecalcitol, maxacalcitol, and paricalcitol;

the phosphate binder is sevelamer or its pharmaceutically acceptable salt; and

the calcium receptor-active compound is cinacalcet or its pharmaceutically acceptable salt.

18) The oral solid pharmaceutical composition of 17) wherein,

the vitamin D is in a dose of 0.1 μg to 100 μg;

the sevelamer is sevelamer hydrochloride or sevelamer carbonate in a dose of 400 mg to 2000 mg; and

the cinacalcet is cinacalcet hydrochloride in a dose of 5 mg to 180 mg.

19) The oral solid pharmaceutical composition of 17) wherein,

the vitamin D is active vitamin D in a dose selected from the group consisting of 0.125 μg, 0.25 μg, 0.5 μg, 1 μg and 2 μg, or is vitamin D in a dose of 1-100 μg;

the sevelamer is sevelamer hydrochloride or sevelamer carbonate in a dose of 400 mg to 800 mg; and

the cinacalcet is cinacalcet hydrochloride in a dose selected from the group consisting of 5 mg, 10 mg, 15 mg, 30 mg, 60 mg, and 90 mg.

20) The oral solid pharmaceutical composition of 16) wherein,

the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

21) A method for preparing the oral solid pharmaceutical compositions of above 1), 6), 11) and 16) comprising the following steps:

granulating cinacalcet and/or sevelamer and/or vitamin D by one of a wet and a dry granulation process, each with at least one pharmaceutically acceptable excipient to form cinacalcet granules and/or sevelamer granules and/or vitamin D granules;

mixing at least two of the cinacalcet granules, sevelamer granules and vitamin D granules to form a granules mixture; and

compressing the granules mixture to tablets or encapsulating the granules mixture into capsules or pulverizing the granules mixture into a dispersion powder.

22) The method for preparing the oral solid pharmaceutical compositions according to above 6), 11) and 16) comprising the following steps:

granulating vitamin D by a wet granulation process with cocoa butter and at least one pharmaceutically acceptable excipient to form vitamin D granules;

drying the vitamin D granules to form dried vitamin D granules;

granulating cinacalcet and/or sevelamer by one of a wet and dry granulation process, each with at least one pharmaceutically acceptable excipient to form cinacalcet granules and/or sevelamer granules;

mixing the dried vitamin D granules with the cinacalcet granules and/or the sevelamer granules to form a granules mixture;

granulating the granules mixture by a wet granulation process with at least one pharmaceutically acceptable excipient to form mixed granules; and

compressing the mixed granules or granules mixture to tablets or encapsulating the mixed granules or granules mixture into capsules.

Embodiments of the present invention include compositions comprising new combinations of active ingredients that advantageously provide proper balances of serum calcium, phosphate and PTH levels. The active ingredient compounds are sevelamer, cinacalcet and vitamin D and the embodiments of the invention include pharmaceutical compositions containing them. The active compounds constituting the combinations are present in the free state or in the form of one of their pharmaceutically acceptable salts.

In the present invention, oral solid compositions enabling sufficient release of active agents can be achieved by combining two or three active compounds, or pharmaceutically acceptable salts thereof, in a single formulation.

As previously stated, “vitamin D” includes vitamin D analogs, such as vitamin D₃ and D₂, and active vitamin D analogs, such as alfacalcidol, calcitriol, falecalcitol, maxacalcitol or paricalcitol, etc. respectively. The pharmacologically/pharmaceutically effective amount of vitamin D is in a range of 1 to 100 μg daily and active vitamin D analogues is in a range of 0.1 μg to 10 μg daily. The daily dosage for cinacalcet is in a range of 30 mg to 180 mg. The daily dosage for sevelamer is 800 mg to 2,400 mg.

An unsolved problem in the field of pharmacology is the difficulty to formulate a combined solid dosage form which comprises two or more active compounds that possess significant differences in their strengths, by simple mixed formulation.

In the inventive compositions comprising pharmacologically effective amounts of vitamin D and the active compounds sevelamer and/or cinacalcet, the content of vitamin D (as an active compound) is much less than other active compounds. The compositions of the present invention are constituted to advantageously provide excellent content uniformity and dissolution.

Accordingly, embodiments of the invention include methods and techniques that solve problems of content uniformity and dissolution by including, for example, the step of mixing separately granulated vitamin D, with the other active compounds. Cinacalcet and/or sevelamer can be added directly or as granules in the mixing step.

The present invention includes a method for producing a vitamin D dispersion or granule which comprises pharmacologically/pharmaceutically effective amounts of vitamin D and at least one pharmaceutically acceptable excipient.

Considering the stability of vitamin D and the immediate dissolution requirement of vitamin D, as well as the practical powder formulation process, the excipient used should have the following characteristics: 1) powder or solid at room temperature; 2) lipid, fat oil or ester; and 3) readily melt once in the body (e.g., at temperature of about 37° C.). While any excipient having the foregoing characteristics can be employed in the present compositions, butters such as cocoa butter and shea butter, have these characteristics and are, for example and without limitation, used as the excipient in the present compositions for dispersing the vitamin D.

The desired properties of the oral solid compositions of the present invention are excellent stability, content uniformity, dissolution, bioavailability and are easy to swallow. In order to have such properties of the combined active compounds of the compositions, the very low strength vitamin D is first dispersed in the suitable excipient, such as cocoa butter which is a vegetable oil, having melting point at 34-38° C. The small amount of active vitamin D can be dissolved into a cocoa butter solution in organic solvent such as dichloromethane or alcohol. This solution can be further dispersed into or granulated with a pharmaceutical excipient or excipient mixture. After drying, the vitamin D, dispersed in solidified cocoa butter, is uniformly mixed with other excipients to form granules. The resulting vitamin D granules are used for further combined formulation with other active compounds or granules containing active compounds, such as sevelamer or/and cinacalcet, or their pharmaceutically acceptable salts. The vitamin D granules prepared in this way are dispersed in the cocoa butter for increased stability. When orally administered, the vitamin D is released immediately after the tablet or capsule disintegrates because the cocoa butter melts in the body.

Cinacalcet or its pharmacologically acceptable salt can be used in the combined formulation either by direct compression, dry granulation or by wet granulation with one or more other pharmaceutical excipients. Wet granulation of the cinacalcet provides a better dissolution profile and the release of the active compound is completed in the targeted time.

Accordingly, cinacalcet hydrochloride is wet granulated using a suitable amount of water with other pharmaceutical inactive excipients, such as hydroxypropyl methylcellulose (HPMC), microcrystalline cellulose (MCC), crospovidone, povidone and silicon dioxide and similar excipients to form a granule which can be used for further combined formulation with other active compounds or granules containing active compounds, such as vitamin D and/or sevelamer or its pharmaceutically acceptable salt.

Sevelamer or its pharmaceutically acceptable salt can be used directly with other granules containing vitamin D and/or cinacalcet, or its pharmaceutically acceptable salt, for compression or encapsulation with optional addition of one or more other suitable pharmaceutical excipients. Sevelamer or its pharmaceutically acceptable salt can also be mixed with other suitable pharmaceutical excipients before formulation with other active compounds or active compound granules.

When the individual active compounds or active granules are prepared accordingly, different combined formulations/compositions with a variety of active strengths can be prepared.

The composition embodiments of the invention include the following combinations:

1) Combination of vitamin D, such as vitamin D₃, alfacalcidol, calcitriol, falecalcitol, maxacalcitol, paricalcitol, and other active forms of vitamin D, and sevelamer or its pharmacologically acceptable salt, such as hydrochloride or carbonate. The oral solid composition comprising the above stated active compounds (vitamin D and sevelamer) or their granules and pharmaceutically acceptable excipient(s).

2) Combination of vitamin D, such as vitamin D₃, alfacalcidol, calcitriol, falecalcitol, maxacalcitol, paricalcitol, and other active forms of vitamin D, and cinacalcet or its pharmaceutically acceptable salt, such as hydrochloride. The oral solid composition comprising the above stated active compounds (vitamin D and cinacalcet) or their granules and pharmaceutically acceptable excipient(s).

3) Combination of cinacalcet and sevelamer or their pharmaceutically acceptable salts, such as hydrochloride or carbonate. The oral solid composition comprising the above stated active compounds (cinacalcet and sevelamer) or their granules and pharmaceutically acceptable excipient(s).

4) Combination of vitamin D, such as vitamin D₃, alfacalcidol, calcitriol, falecalcitol, maxacalcitol, paricalcitol, and other active forms of vitamin D, cinacalcet and sevelamer or their pharmaceutically acceptable salts, such as hydrochloride or carbonate. The oral solid composition comprising the above stated active compounds (vitamin D, cinacalcet and sevelamer) or their granules and pharmaceutically acceptable excipient(s).

The composition combinations according to the invention are pharmaceutical compositions for administration to mammals, including humans, for the treatment of kidney diseases, especially chronic kidney disease. The composition combinations according to the invention are pharmaceutical compositions for administration to mammals, including humans, for the treatment of mineral bone disorder, especially chronic kidney disease—mineral bone disorder. The composition combinations according to the invention that include cinacalcet are pharmaceutical compositions for administration to mammals, including humans, for the treatment of hypercalcemia in patients with parathyroid carcinoma.

With respect to the following description of the compositions' active compound dosages, the quantities of vitamin D, sevelamer and cinacalcet or their pharmaceutically acceptable salts are expressed as vitamin D and cinacalcet in free form; sevelamer in salt form.

When the compositions of the invention are administered in humans by the oral route, it is preferable that the daily dose of sevelamer hydrochloride or carbonate is between 400 and 2,400 mg, the daily dose of cinacalcet is preferably between 5 mg and 180 mg, the daily dose of vitamin D is between 0.1 μg to 100 μg.

When the compositions of the invention are administered in humans by the parenteral route, it is preferable that the daily dose of cinacalcet is between 5 mg and 180 mg, and the daily dose of vitamin D is between 0.1 μg to 100 μg.

The compositions of the invention comprise vitamin D, sevelamer and cinacalcet in the preferable amounts stated below:

1) The combined compositions containing sevelamer and cinacalcet having doses of 400 mg to 2000 mg of sevelamer hydrochloride or sevelamer carbonate, 5 mg to 180 mg of cinacalcet hydrochloride, whereby the therapeutic effect of the compositions is for the treatment of chronic kidney disease. More preferable compositions have dose portions of 400 mg or 800 mg of sevelamer hydrochloride or sevelamer carbonate, combined with 5 mg, 10 mg, 15 mg, 30 mg, 60 mg or 90 mg of cinacalcet, respectively. A most preferable formulation is 800 mg of sevelamer hydrochloride and 30 mg of cinacalcet which is present as cinacalcet hydrochloride.

2) The combined compositions containing vitamin D and cinacalcet having doses of 0.1 μg to 10 μg of active vitamin D or 1 μg to 100 μg vitamin D, 5 mg to 180 mg of cinacalcet hydrochloride, whereby the therapeutic effect of the compositions is for the treatment of chronic kidney disease. More preferable compositions have dose portions of 0.125 μg, 0.25 μg, 0.5 μg, 1 μg and 2 μg of active vitamin D or 1 μg, 5 μg, 10 μg, 20 μg, 50 μg and 100 μg of vitamin D, combined with 5 mg, 10 mg, 15 mg, 30 mg, 60 mg and 90 mg of cinacalcet hydrochloride, respectively. A most preferable formulation is 1 μg of vitamin D and 30 mg of cinacalcet which is present as cinacalcet hydrochloride.

3) The combined compositions containing sevelamer and vitamin D having doses of 400 mg to 2000 mg of sevelamer hydrochloride or sevelamer carbonate, 0.1 μg to 100 μg of active vitamin D, whereby the therapeutic effect of the compositions is for the treatment of chronic kidney disease. More preferable compositions have dose portions of 400 mg and 800 mg of sevelamer hydrochloride or sevelamer carbonate, combined with 0.125 μg, 0.25 μg, 0.5 μg, 1 μg and 2 μg of active vitamin D or 1 μg, 5 μg, 10 μg, 20 μg, 50 μg and 100 μg of vitamin D, respectively. A most preferable formulation is 800 mg of sevelamer hydrochloride and 1 μg of vitamin D.

4) The combined compositions containing sevelamer, cinacalcet and vitamin D having doses of 400 mg to 2000 mg of sevelamer hydrochloride or sevelamer carbonate, 5 mg to 180 mg of cinacalcet hydrochloride and 0.1 μg to 100 μg of vitamin D or 0.1 μg to 10 μg active vitamin D, whereby the therapeutic effect of the compositions is for the treatment of chronic kidney disease. More preferable compositions have dose portions of 400 mg and 800 mg of sevelamer hydrochloride or sevelamer carbonate, combined with 5 mg, 10 mg, 15 mg, 30 mg, 60 mg and 90 mg of cinacalcet hydrochloride respectively and 0.125 μg, 0.25 μg, 0.5 μg, 1 μg and 2 μg of active vitamin D or 1 μg, 5 μg, 10 μg, 20 μg, 50 μg and 100 μg of vitamin D, respectively. A most preferable formulation is 800 mg of sevelamer hydrochloride, 30 mg of cinacalcet which is present as cinacalcet hydrochloride and 1 μg of vitamin D.

The combined compositions can be formulated by a wet granulation process, or dry granulation process, or combination of the wet granulation and dry granulation processes to either compress the compositions to tablets or encapsulate the compositions into capsules. The following are exemplary methods of preparing compositions of the present invention.

1) A composition formulation according to any one of aforementioned compositions wherein vitamin D or active vitamin D is a constituent, is prepared by the following formulation process:
a) vitamin D is wet granulated with a pharmaceutically acceptable butter such as a cocoa butter and at least one pharmaceutically acceptable excipient, followed by drying the granules;
b) combine and mix the dried granules with sevelamer salt and/or cinacalcet hydrochloride which is either granulated by a wet granulation or by direct mix with at least one pharmaceutically acceptable excipient.
c) compress the combined granules to tablets or encapsulate into capsules.

2) A composition formulation according to any one of aforementioned compositions wherein cinacalcet hydrochloride is a constituent, is prepared by wet granulation or a direct mixing process. The wet granulation process is carried out as follows:
a) cinacalcet hydrochloride is wet granulated with at least one pharmaceutically acceptable excipient, followed by drying the granule.
b) combine and mix the dried granules with sevelamer salt or/and vitamin D (or active vitamin D) granules with optional addition of suitable pharmaceutically acceptable excipient(s).
c) compress the combined granules/mixture to tablets or encapsulate into capsules.

3) A composition formulation according to any one of aforementioned compositions wherein sevelamer hydrochloride or carbonate is a constituent, is prepared by direct mixing process or is wet granulated with cinacalcet hydrochloride, and/or vitamin D (or active vitamin D) granules, with optional addition of suitable pharmaceutically acceptable excipient(s).

The oral solid compositions of the present invention comprise a pharmaceutically acceptable amount of sevelamer hydrochloride or carbonate, cinacalcet hydrochloride and/or vitamin D (or active vitamin D), and comprising at least one pharmaceutically acceptable excipient being sugar alcohol such as starch and lactose, or cellulose derivatives such as microcrystalline cellulose and hydroxypropyl methylcellulose, or povidone and its derivatives such as copovidone and crospovidone, or lubricant such as magnesium stearate and colloidal silicon dioxide etc.

The aforementioned composition formulations are made with constituent active compound doses for the reduction of parathyroid hormone (PTH), serum calcium, phosphorus, and calcium-phosphate product levels, especially in patients with chronic kidney disease (CKD) on dialysis. It is also for the treatment of hypercalcemia in patients with parathyroid carcinoma.

In the pharmaceutical compositions of the present invention for oral administration, the active ingredients may be administered in unit forms, mixed with conventional pharmaceutical carriers, to patients. The appropriate unit forms for administration comprise the forms for oral administration such as tablets, gelatin capsules, powders, granules and oral solutions or suspensions.

The pharmaceutical compositions of the present invention containing vitamin D and cinacalcet or its pharmacologically acceptable salt may be administrated with conventional pharmaceutical carriers, as the forms for sublingual or buccal administration, the forms for subcutaneous, intramuscular, intravenous, intranasal or intraocular administration.

When a solid composition in the form of tablets is prepared, the tablets can be coated with appropriate materials or alternatively they can be treated so that they have a prolonged or delayed activity and that they continuously liberate a predetermined quantity of active ingredient compound.

A preparation in the form of gelatin capsules is obtained by mixing an active ingredient compound with a diluent and by pouring the mixture obtained into soft or hard gelatin capsules.

A preparation in syrup or elixir form may contain an active ingredient compound together with a sweetener, preferably calorie free, methyl paraben or propylparaben as antiseptic, as well as a taste enhancer and an appropriate coloring.

Water-dispersible granules or powders may contain an active ingredient compound mixed with dispersing agents or wetting agents, or suspending agents, such as polyvinylpyrrolidone, as well as sweeteners or flavor correctors.

For parenteral, intranasal or intraocular administration of the present pharmaceutical compositions, aqueous suspensions, isotonic saline solutions, sterile and injectable solutions are used which contain dispersing agents and/or wetting agents which are pharmacologically compatible.

The composition active ingredient compounds can also be formulated in the form of microcapsules, optionally with one or more carriers or additives.

The active ingredient compounds of the inventive compositions can also be provided in the form of a complex with cyclodextrin, for example α-, β- or γ-cyclodextrin, 2-hydroxypropyl-β-cyclodextrin or methyl-β-cyclodextrin.

The present invention is demonstrated with reference to the following examples, which are of an illustrative nature only and which are to be construed as non-limiting.

### EXAMPLES

### Example 1

### Preparation of Vitamin D (Alfacalcidol) Dispersion

a) 3 g of cocoa butter were dissolved in 20 ml of dichloromethane at room temperature, and a solution of vitamin E, BHA and BHT (30 mg of each) in ethanol was added, and stirred until mixed well.

b) About 2.92 mg of alfacalcidol was dissolved in an amber volumetric flask with about 5 ml of ethanol. This solution was gradually added into the mixed solution of cocoa butter, the alfacalcidol flask was rinsed with about 5 ml of ethanol, and then it was added into the mixed solution. This mixed solution was stirred until a homogenous solution was obtained.

c) A pre-mixed excipient comprised of 14.3 g of HPMC (E5) and 128.2 g of MCC (PH112) was prepared in a small granulator, the mixed solution prepared in b) was gradually (in 1-3 min) added into the pre-mixed excipient while stirred vigorously until the mixture uniformity was satisfactory (about 1-5 min with high shear granulator).

d) The wet granules were passed through a 40 mesh sieve before drying in the vacuum dryer at temperature of about 30° C. to remove the residual solvents.

e) The dried alfacalcidol (about 1 μg alfacalcidol/50 mg granule) granule was passed through a 40 mesh sieve and packaged with polyethylene (PE), and further wrapped on the outside with a black plastic bag for further usage.

### Example 2

Preparation of combined formulation of sevelamer hydrochloride and alfacalcidol (vitamin D) shown in Table 3.

TABLE 3
Composition for 800 mg Sevelamer HCl and 1 μg Alfacalcidol Tablet

| Ingredient | Weight (mg)/tablet |
|---|---|
| Sevelamer HCl | 800 |
| Alfacalcidol granules | 50 |
| Copovidone | 20 |
| Crospovidone | 20 |
| Colloidal silicon dioxide | 5 |
| Magnesium stearate | 5 |
| Total weight | 900 |

According to the composition shown in Table 3, all materials were weighed out and then passed through a 20 mesh sieve. The alfacalcidol granules, copovidone, crospovidone and colloidal silicon dioxide were mixed, and sevelamer HCl was then added into the mixture and further mixed. Magnesium stearate was next added and mixed to give a final blend. The final blend was compressed to tablets with 900 mg nominal weight. Each tablet contained 800 mg of sevelamer HCl and 1 μg of alfacalcidol.

### Example 3

Preparation of combined formulation of sevelamer hydrochloride and cinacalcet hydrochloride shown in Table 4.

TABLE 4
Composition for 800 mg Sevelamer HCl and 30 mg Cinacalcet Tablet

| Ingredient | Weight (mg)/Tablet |
|---|---|
| Sevelamer HCl | 800 |
| Cinacalcet HCl | 33* |
| Copovidone | 30 |
| Crospovidone | 10 |
| Colloidal silicon dioxide | 3 |
| Magnesium stearate | 4 |
| Total weight | 880 |

*33 mg cinacalcet HCl is equivalent to 30 mg cinacalcet free base

According to the composition shown in Table 4, all materials were weighed out and then passed through 20 mesh sieve. Cinacalcet, copovidone, crospovidone and colloidal silicon dioxide were mixed and then sevelamer HCl was added and mixed. Magnesium stearate was added and mixed to give a final blend. The final blend was compressed to tablets with 880 mg nominal weight. Each tablet contained 800 mg of sevelamer HCl and 30 mg of cinacalcet.

### Example 4

Preparation of combined formulation of Cinacalcet HCl and Alfacalcidol (vitamin D) shown in Table 5.

TABLE 5
Composition for 30 mg Cinacalcet and 1 μg Alfacalcidol Tablet

| Ingredient | Weight (mg)/Tablet |
|---|---|
| Cinacalcet HCl | 33* |
| Alfacalcidol granules | 50 |
| Copovidone | 40 |
| Crospovidone | 15 |
| Microcrystalline cellulose (PH112) | 10 |
| Partial pregelatinized starch | 48 |
| Colloidal silicon dioxide | 2 |
| Magnesium stearate | 2 |
| Total weight | 200 |

*33 mg cinacalcet HCl is equivalent to 30 mg cinacalcet free base

According to the composition shown in Table 5, all materials were weighed out and then passed through a 20 mesh sieve. Cinacalcet HCl, alfacalcidol granules, copovidone, crospovidone, microcrystalline cellulose (MCC), partial pregelatinized starch 1500 and colloidal silicon dioxide were mixed, and then magnesium stearate was added and mixed to give a final blend. The final blend was compressed to tablets with 200 mg nominal weight. Each tablet contained 30 mg of cinacalcet and 1 μg of alfacalcidol.

### Example 5

Preparation of combined formulation of sevelamer HCl, cinacalcet HCl and alfacalcidol (vitamin D) shown in Table 6.

TABLE 6
Composition for 800 mg Sevelamer HCl, 30 mg Cinacalcet and 1 μg
Alfacalcidol Tablet

| Ingredient | Weight (mg)/Tablet |
|---|---|
| Sevelamer HCl | 800 |
| Cinacalcet HCl | 33* |
| Alfacalcidol granule | 50 |
| Copovidone | 30 |
| Crospovidone | 15 |
| Colloidal silicon dioxide | 5 |
| Magnesium stearate | 7 |
| Total weight | 940 |

*33 mg cinacalcet HCl is equivalent to 30 mg cinacalcet free base

According to the composition shown in Table 6, all materials were weighed out and then passed through a 20 mesh sieve. Cinacalcet HCl, alfacalcidol granule, copovidone, crospovidone and colloidal silicon dioxide were mixed, then sevelamer HCl was added and mixed. Magnesium stearate was added and mixed to give a final blend. The final blend was compressed to tablets with 940 mg nominal weight. Each tablet contained 800 mg of sevelamer HCl, 30 mg of cinacalcet, and 1 μg of alfacalcidol.

### Example 6

Formulation of cinacalcet tablets with about 16.5% drug loading by a direct compression process shown in Table 7.

TABLE 7
Composition for 30 mg Cinacalcet Tablet
with about 16.5% Drug Loading

| Ingredient | Weight (mg)/Tablet |
|---|---|
| Cinacalcet HCl | 33* |
| Copovidone | 40 |
| Crospovidone | 15 |
| Microcrystalline cellulose | 48 |
| Partial pregelatinized starch | 60 |
| Colloidal silicon dioxide | 2 |
| Magnesium stearate | 2 |
| Total weight | 200 |

*33 mg cinacalcet HCl is equivalent to 30 mg cinacalcet free base

According to the composition shown in Table 7, all materials were weighed out and then passed a through 40 mesh sieve. Cinacalcet HCl, copovidone, crospovidone, microcrystalline cellulose (MCC), partial pregelatinized starch 1500 and colloidal silicon dioxide were mixed, and then magnesium stearate was added and mixed to give a final blend. The final blend was compressed to tablets with 200 mg nominal weight. Each tablet contains 30 mg of cinacalcet.

The tablets prepared from above examples 3 to 6 were subjected to dissolution tests using USP 2 (paddle) apparatus in 900 mL of 0.05 N HCl with 75 rpm agitation. The dissolution results of cinacalcet are summarized in Table 8.

TABLE 8
Dissolution Results of Tablets from Example 3 to 6

| Time | Example 3 | Example 4 | Example 5 | Example 6 |
|---|---|---|---|---|
| (min) | (%) | (%) | (%) | (%) |
| 10 | 62 | 48 | 70 | 42 |
| 15 | 81 | 61 | 85 | 61 |
| 20 | 88 | 69 | 90 | 70 |
| 30 | 93 | 74 | 94 | 75 |

### Example 7

Formulation of cinacalcet tablets with about 11% drug loading by a direct compression process shown in Table 9.

TABLE 9
Composition for 30 mg and 90 mg Cinacalcet
Tablets with about 11% Drug Loading

| | Weight (mg)/Tablet | |
|---|---|---|
| Ingredient | 30 mg | 90 mg |
| Cinacalcet HCl | 33* | 99* |
| Copovidone | 40 | 120 |
| Crospovidone | 30 | 90 |
| Microcrystalline cellulose | 70 | 210 |
| Partial pregelatinized starch | 120 | 360 |
| Colloidal silicon dioxide | 5 | 15 |
| Magnesium stearate | 2 | 6 |
| Total weight | 300 | 900 |

*33 mg and 99 mg cinacalcet HCl are equivalent to 30 mg and 90 mg
cinacalcet free base, respectively

According to the compositions shown in Table 9, all materials were weighed out and then passed through 40 mesh sieve. Cinacalcet HCl, copovidone, crospovidone, microcrystalline cellulose (MCC), partial pregelatinized starch 1500 and colloidal silicon dioxide were mixed, and then magnesium stearate was added and mixed to give a final blend. The final blend was compressed to tablets with 300 mg nominal weight in which each tablet contained 30 mg of cinacalcet, and to tablets with 900 mg nominal weight in which each tablet contained 90 mg of cinacalcet.

The tablets prepared from Example 7 were subjected to dissolution tests using USP 2 (paddle) apparatus in 900 mL of 0.05 N HCl with 75 rpm agitation. The dissolution profiles of cinacalcet are summarized in Table 10.

TABLE 10
Dissolution Results of Tablets from Example 7

| | Example 7 (% dissolved) | |
|---|---|---|
| Time (min) | 30 mg | 90 mg |
| 10 | 90 | 73 |
| 15 | 90 | 85 |
| 20 | 90 | 85 |
| 30 | 89 | 85 |
| 45 | 90 | 85 |

### Example 8

Formulation of cinacalcet HCl granules by wet granulation process shown in Table 11.

TABLE 11
Composition for 30 mg, 60 mg, and 90 mg Cinacalcet Tablet

| | Weight (mg)/Tablet | | |
|---|---|---|---|
| Ingredient | 30 mg cinacalcet | 60 mg cinacalcet | 90 mg cinacalcet |
| Cinacalcet HCl | 33* | 66* | 99* |
| Povidone | 8 | 16 | 24 |
| Crospovidone | 3 | 6 | 9 |
| Microcrystalline | 69 | 138 | 207 |
| cellulose | | | |
| Hyperomellose | 10 | 20 | 60 |
| Colloidal silicon | 1 | 2 | 3 |
| dioxide | | | |
| Total weight | 124 | 248 | 372 |

*33 mg, 66 mg, and 99 mg cinacalcet HCl is equivalent to 30 mg , 60 mg, and
90 mg cinacalcet free base, respectively

According to the compositions shown in Table 11, all materials were weighed out and then passed through a 40 mesh sieve. Cinalcalcet HCl, povidone, hyperomellose and part of microcrystalline cellulose (MCC) were wet granulated with purified water (20-30% w/w of mixture) until unified into granules. The granules were dried at 50° C. and then passed through a 40 mesh sieve.

The above resulting granules were dry mixed with MCC, crospovidone, colloidal silicon dioxide and magnesium stearate to yield a final blend. The final blend was compressed to tablets containing 30 mg, 60 mg, and 90 mg cinacalcet by adjusting tablet weights.

The tablets prepared from Example 8 were subjected to dissolution tests using USP 2 (paddle) apparatus in 900 mL of 0.05 N HCl with 75 rpm agitation. The dissolution profiles of cinacalcet are summarized in Table 12.

TABLE 12
Dissolution Results of Tablets from Example 8

| | Example 8 (% dissolved) | | |
|---|---|---|---|
| Time (min) | 30 mg | 60 mg | 90 mg |
| 10 | 82 | 80 | 82 |
| 15 | 94 | 87 | 90 |
| 20 | 100 | 92 | 94 |
| 30 | 100 | 96 | 94 |
| 45 | 101 | 98 | 96 |

### Example 9

Combined formulations comprised of cinacalcet hydrochloride wet granules and/or alfacalcidol granules and/or sevelamer hydrochloride shown in Table 13.

TABLE 13
Composition for Tablet having
Combinations of Different Active Compounds

| | Weight (mg)/Tablet | | | |
|---|---|---|---|---|
| | | | Sevelamer | |
| | Sevelamer | Sevelamer | cinacalcet | Alfacalcidol |
| | alfacalcidol | cinacalcet | alfacalcidol | cinacalcet |
| Ingredient | Tablet | Tablet | Tablet | Tablet |
| Sevelamer HCl | 800 | 800 | 800 | 0 |
| Alfacalcidol | 50 | 0 | 50 | 50 |
| granule | | | | |
| Cinacalcet HCl | 0 | 124 | 124 | 124 |
| granule | | | | |
| Microcrystalline | 0 | 28 | 0 | 68 |
| cellulose | | | | |
| Copovidone | 30 | 30 | 30 | 0 |
| Colloidal | 5 | 4 | 3 | 1 |
| silicon dioxide | | | | |
| Crospovidone | 10 | 10 | 10 | 6 |
| Magnesium | 5 | 4 | 3 | 1 |
| stearate | | | | |
| Total | 900 | 1000 | 1020 | 250 |

According to the four compositions shown in Table 13, all materials were weighed out and then passed through a 40 mesh sieve respectively. The active and/or active granules for each composition were mixed with corresponding excipients, and magnesium stearate was added and mixed to give the four final blends. The final blends were compressed to tablets.

The tablets containing cinacalcet prepared from the Example 9 were subjected to dissolution tests using USP 2 (paddle) apparatus in 900 mL of 0.05 N HCl with 75 rpm agitation. The dissolution profiles of cinacalcet are summarized in Table 14.

TABLE 14
Dissolution Results of Tablets from Example 9

| | Example 9 (% dissolved) | | |
|---|---|---|---|
| | | Sevelamer | |
| | Sevelamer | cinacalcet | Alfacalcidol |
| | cinacalcet | alfacalcidol | cinacalcet |
| Time (min) | Tablet | Tablet | Tablet |
| 10 | 91 | 96 | 88 |
| 15 | 98 | 101 | 102 |
| 20 | 100 | 102 | 101 |
| 30 | 102 | 103 | 103 |
| 45 | 104 | 108 | 102 |

### Example 10

### Preparation of Vitamin D₃ (VD₃) Dispersion

a) 3 g of cocoa butter were dissolved in 20 ml of dichloromethane at room temperature, and a solution of vitamin E, BHA and BHT (30 mg of each) in 5 ml of ethanol was added, and stirred until mixed well.

b) 75 mg of vitamin D₃ were dissolved in an amber volumetric flask with about 5 ml of ethanol. The solution was added gradually into the mixed solution of cocoa butter; the vitamin D₃ flask was rinsed with about 5 ml of ethanol; and then the rinse was added into the mixed solution. The mixed solution was stirred until a homogenous solution is obtained.

c) A pre-mixed excipient comprised of 17 g of HPMC (E5) and 130 g of MCC (PH112) was prepared in a small granulator, the mixed solution prepared in b) was gradually (in 1-3 min) added into the pre-mixed excipient while stirring vigorously until the mixture uniformity was satisfactory (about 1-5 min with high share granulator).

d) The wet granules from c) were passed through a 40 mesh sieve before drying in a vacuum dryer at a temperature about 30° C. to remove the residual solvents.

e) The dried vitamin D₃ (about 25 μg vitamin D₃/50 mg granule) granules were passed through a 40 mesh sieve and packaged with polyethylene (PE), and further wrapped on the outside with a black plastic bag for further usage. The composition for 25 μg vitamin D₃/50 mg solid dispersion granules is shown in Table 15.

TABLE 15
Composition for 25 μg vitamin D₃/50 mg solid dispersion granules

| Item | Weight |
|---|---|
| Cocoa Butter | 3 g |
| BHA | 30 mg |
| BHT | 30 mg |
| Vitamin E | 30 mg |
| Vitamin D3 | 75 mg |
| HPMC (E5) | 17 g |
| MCC (PH112) | 130 g |
| Total | 150 g |

### Example 11

Using similar formulation procedures to those employed in Examples 2, 3, 4 and 5, tablets were prepared based on the following compositions a, b, c and d respectively.

TABLE 16
Composition for 800 mg Sevelamer HCl and 25 μg vitamin D₃ Tablet
Composition a

| Ingredient | Weight (mg)/Tablet |
|---|---|
| Sevelamer | 800 |
| Solid dispersion | 50 (contains 25 μg VD₃) |
| (contains VD₃) | |
| MCC (PH101) | 124 |
| Copovidone VA64 fine | 30 |
| Crospovidone CL | 10 |
| Colloidal silicon dioxide | 3 |
| Magnesium stearate | 3 |
| Total weight | 1020 |

TABLE 17
Composition for 800 mg Sevelamer HCl and 30 mg Cinacalcet Tablet
Composition b

| Ingredient | Weight (mg)/Tablet |
|---|---|
| Sevelamer | 800 |
| Cinacalcet HCl | 33 |
| HPMC E5 | 10 |
| Povidone K30 | 8 |
| MCC (PH101) | 97 |
| Crospovidone CL | 13 |
| Copovidone VA64 fine | 30 |
| Colloidal silicon dioxide | 5 |
| Magnesium stearate | 4 |
| Total weight | 1000 |

TABLE 18
Composition for 30 mg Cinacalcet and 25 μg Vitamin D₃ Tablet
Composition c

| Ingredient | Weight (mg)/Tablet |
|---|---|
| Solid dispersion (contains VD₃) | 50 (contains 25 μg VD₃) |
| Cinacalcet HCl | 33 |
| HPMC E5 | 10 |
| Povidone K30 | 8 |
| MCC (PH101) | 137 |
| Crospovidone CL | 9 |
| Colloidal silicon dioxide | 2 |
| Magnesium stearate | 1 |
| Total weight | 250 |

TABLE 19
Composition for 800 mg Sevelamer HCl, 30 mg Cinacalcet and 25 μg
Vitamin D₃ Tablet
Composition d

| Ingredient | Weight (mg)/Tablet |
|---|---|
| Sevelamer | 800 |
| Solid dispersion (contains VD₃) | 50 (contains 25 μg VD₃) |
| Cinacalcet HCl | 33 |
| HPMC E5 | 10 |
| Povidone K30 | 8 |
| MCC (PH101) | 69 |
| Crospovidone CL | 13 |
| Copovidone VA64 fine | 30 |
| Colloidal silicon dioxide | 4 |
| Total weight | 1020 |

Tablets resulting from above compositions a, b and d were tested for the binding of phosphate, measured as the concentration of phosphate ions. The starting concentration of KH₂PO₄ in the dissolution media was 2.722 mg/ml. The test results are shown in Table 20.

TABLE 20
Phosphate Binding Results of Tablets from compositions a, b and d
(measuring the concentration of phosphate ion)
Starting concentration of KH₂PO₄ in dissolution media: 2.722 mg/ml

| | Composition d | Composition a | Composition b |
|---|---|---|---|
| | phosphate ion | phosphate ion | phosphate ion |
| Time | (mg/ml) | (mg/ml) | (mg/ml) |
| 10 min | 1.661 | 1.666 | 1.694 |
| 20 min | 1.636 | 1.629 | 1.650 |
| 30 min | 1.454 | 1.527 | 1.561 |
| 45 min | 1.402 | 1.463 | 1.482 |
| 60 min | 1.339 | 1.387 | 1.494 |

Tablets resulting from the above compositions a, c and d were subjected to dissolution tests, measuring the % concentration of Vitamin D₃ in 500 ml of dissolution media. The dissolution results are shown in Table 21.

TABLE 21
Dissolution Results of Tablets from above compositions a, c and d
(measuring the % concentration of Vitamin D₃ in 500 ml of dissolution media)

| Time | Composition d | Composition a | Composition c |
|---|---|---|---|
| 30 min | 103.2 | 89.6 | 87.2 |

While only a few exemplary embodiments of this invention have been described in detail, those skilled in the art will recognize that there are many possible variations and modifications which may be made in the exemplary embodiments while yet retaining many of the novel and advantageous features of this invention. Accordingly, it is intended that the following claims cover all such modifications and variations.

1. An oral solid pharmaceutical composition for the treatment of kidney diseases and mineral bone disorder comprising:
a phosphate binder;
a calcium receptor-active compound; and
at least one pharmaceutically acceptable excipient.

2. The oral solid pharmaceutical composition of claim 1 wherein,
the phosphate binder is sevelamer or its pharmaceutically acceptable salt; and
the calcium receptor-active compound is cinacalcet or its pharmaceutically acceptable salt.

3. The oral solid pharmaceutical composition of claim 2 wherein,
the sevelamer is sevelamer hydrochloride or sevelamer carbonate in a dose of 400 mg to 2000 mg; and
the cinacalcet is cinacalcet hydrochloride in a dose of 5 mg to 90 mg.

4. The oral solid pharmaceutical composition of claim 2 wherein,
the sevelamer is sevelamer hydrochloride or sevelamer carbonate in a dose of 400 mg or 800 mg; and
the cinacalcet is cinacalcet hydrochloride in a dose selected from the group consisting of 5 mg, 10 mg, 15 mg, 30 mg, 60 mg, and 90 mg.

5. The oral solid pharmaceutical composition of claim 1 wherein,
the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

6. An oral solid pharmaceutical composition for the treatment of kidney diseases and mineral bone disorder comprising:
vitamin D;
a calcium receptor-active compound; and
at least one pharmaceutically acceptable excipient.

7. The oral solid pharmaceutical composition of claim 6 wherein,
the calcium receptor-active compound is cinacalcet or its pharmaceutically acceptable salt; and
the vitamin D is vitamin D or vitamin D₃, D₂ or active vitamin D selected from the group consisting of alfacalcidol, calcitriol, falecalcitol, maxacalcitol, and paricalcitol.

8. The oral solid pharmaceutical composition of claim 7 wherein,
the cinacalcet is cinacalcet hydrochloride in a dose of 5 mg to 180 mg; and
the vitamin D is in a dose of 0.1 μg to 100 μg.

9. The oral solid pharmaceutical composition of claim 7 wherein,
the cinacalcet is cinacalcet hydrochloride in a dose selected from the group consisting of 30 mg, 60 mg and 90 mg; and
the vitamin D is active vitamin D in a dose selected from the group consisting of 0.125 μg, 0.25 μg, 0.5 μg, 1 μg and 2 μg, or is vitamin D in a dose of 1-100 μg.

10. The oral solid pharmaceutical composition of claim 6 wherein,
the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

11. An oral solid pharmaceutical composition for the treatment of kidney diseases and mineral bone disorder comprising:
vitamin D;
a phosphate binder; and
at least one pharmaceutically acceptable excipient.

12. The oral solid pharmaceutical composition of claim 11 wherein,
the phosphate binder is sevelamer or its pharmaceutically acceptable salt; and
the vitamin D is vitamin D or vitamin D₃, D₂ or active vitamin D selected from the group consisting of alfacalcidol, calcitriol, falecalcitol, maxacalcitol, and paricalcitol.

13. The oral solid pharmaceutical composition of claim 12 wherein,
the sevelamer is sevelamer hydrochloride or sevelamer carbonate in a dose of 400 mg to 2000 mg; and
the vitamin D is in a dose of 0.1 μg to 100 μg.

14. The oral solid pharmaceutical composition of claim 12 wherein,
the sevelamer is sevelamer hydrochloride or sevelamer carbonate in a dose of 400 mg to 800 mg; and
the vitamin D is active vitamin D in a dose selected from the group consisting of 0.125 μg, 0.25 μg, 0.5 μg, 1 μg and 2 μg, or is vitamin D in a dose of 1-100 μg.

15. The oral solid pharmaceutical composition of claim 11 wherein,
the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

16. An oral solid pharmaceutical composition for the treatment of kidney diseases and mineral bone disorder comprising:
vitamin D;
a phosphate binder;
a calcium receptor-active compound; and
at least one pharmaceutically acceptable excipient.

17. The oral solid pharmaceutical composition of claim 16 wherein,
the vitamin D is vitamin D, vitamin D₃, D₂ or active vitamin D selected from the group consisting of alfacalcidol, calcitriol, falecalcitol, maxacalcitol, and paricalcitol;
the phosphate binder is sevelamer or its pharmaceutically acceptable salt; and
the calcium receptor-active compound is cinacalcet or its pharmaceutically acceptable salt.

18. The oral solid pharmaceutical composition of claim 17 wherein,
the vitamin D is in a dose of 0.1 μg to 100 μg;
the sevelamer is sevelamer hydrochloride or sevelamer carbonate in a dose of 400 mg to 2000 mg; and
the cinacalcet is cinacalcet hydrochloride in a dose of 5 mg to 180 mg.

19. The oral solid pharmaceutical composition of claim 17 wherein,
the vitamin D is active vitamin D in a dose selected from the group consisting of 0.125 μg, 0.25 μg, 0.5 μg, 1 μg and 2 μg, or is vitamin D in a dose of 1-100 μg;
the sevelamer is sevelamer hydrochloride or sevelamer carbonate in a dose of 400 mg to 800 mg; and
the cinacalcet is cinacalcet hydrochloride in a dose selected from the group consisting of 5 mg, 10 mg, 15 mg, 30 mg, 60 mg, and 90 mg.

20. The oral solid pharmaceutical composition of claim 16 wherein,
the pharmaceutically acceptable excipient is a sugar alcohol selected from the group consisting of starch and lactose, or a cellulose derivative selected from the group consisting of microcrystalline cellulose and hydroxypropyl methylcellulose, or povidone or povidone derivatives selected from the group consisting of copovidone and crospovidone.

21. A method for preparing the oral solid pharmaceutical compositions of claim 1, 6, 11 or 16 comprising the following steps:
granulating cinacalcet and/or sevelamer and/or vitamin D by one of a wet and a dry granulation process, each with at least one pharmaceutically acceptable excipient to form cinacalcet granules and/or sevelamer granules and/or vitamin D granules;
mixing at least two of the cinacalcet granules, sevelamer granules and vitamin D granules to form a granules mixture; and
compressing the granules mixture to tablets or encapsulating the granules mixture into capsules or pulverizing the granules mixture into a dispersion powder.

22. The method for preparing the oral solid pharmaceutical compositions according to claim 6, 11 or 16 comprising the following steps:
granulating vitamin D by a wet granulation process with cocoa butter and at least one pharmaceutically acceptable excipient to form vitamin D granules;
drying the vitamin D granules to form dried vitamin D granules;
granulating cinacalcet and/or sevelamer by one of a wet and dry granulation process, each with at least one pharmaceutically acceptable excipient to form cinacalcet granules and/or sevelamer granules;
mixing the dried vitamin D granules with the cinacalcet granules and/or the sevelamer granules to form a granules mixture;
granulating the granules mixture by a wet granulation process with at least one pharmaceutically acceptable excipient to form mixed granules; and
compressing the mixed granules or granules mixture to tablets or encapsulating the mixed granules or granules mixture into capsules.
